(19) United States
(12) Patent Application Publication
(10) Pub. No.: US 2026/0305957 A1
(43) Pub. Date: Oct. 8, 2026

(54) Predicting probability of developing preeclampsia

(71) Applicant: 80 Beats Medical GmbH, Berlin (DE)

(72) Inventors: Vijay IYENGAR, Ludwigsfelde (DE); Jörg HEISE, Michendorf (DE)

(21) Appl. No.: 19/639,268

(22) Filed: Apr. 5, 2026

Related U.S. Application Data
(60) Provisional application No. 63/783,296, filed on Apr. 4, 2025.

(51) Int. Cl. A61B 5/0205; A61B 5/00; A61B 5/01; A61B 5/02; A61B 5/022; A61B 5/0295; A61B 5/1455

(52) U.S. Cl. CPC A61B 5/02055; A61B 5/01; A61B 5/02007; A61B 5/02028; A61B 5/02225; A61B 5/0295; A61B 5/14551; A61B 5/7264; A61B 5/7271; A61B 5/742

(57) ABSTRACT

The present disclosure relates to a system (100) and a method (200) for predicting a cardiovascular condition in a subject (106) during pregnancy. The system (100) has a control unit (104). The control unit (104) receives one or more physiological waveform data and a historical medical data of the subject (106). The control unit (104) derives a plurality of cardiovascular parameters based on an analysis of the received physiological waveform data, processes the derived cardiovascular parameters based on a trained predictive model and generates one or more cardiovascular risk scores (110) based on the processing, thereby predicting the cardiovascular condition in the subject (106) during pregnancy.

### CROSS-REFERENCE TO RELATED APPLICATIONS

This application claims benefit of U.S. provisional patent application Ser. No. 63/783,296, filed Apr. 4, 2025, which is herein incorporated by reference in its entirety.

### FIELD OF THE INVENTION

The present invention relates to cardiovascular conditions. More particularly, the present invention relates to a system and a method for predicting a cardiovascular condition in a subject during pregnancy.

### BACKGROUND OF THE INVENTION

Preeclampsia (PE) and Gestational Hypertension (GH) are common medical complications arising during pregnancy. PE is characterized by hypertension and excess protein excretion in urine, while GH is defined as new-onset hypertension without proteinuria or end-organ dysfunction associated with PE. Together, these hypertensive disorders of pregnancy affect a significant proportion of pregnancies worldwide in which PE affects at least 3-6% of all pregnancies. Both PE and GH conditions are leading causes of maternal and fetal morbidity and mortality. PE alone is responsible for approximately 76,000 maternal and 500,000 infant deaths per year. The symptoms of both the conditions typically arise after the 20th week of gestation. PE is multifactorial; most cases of PE are exemplified by abnormal maternal uterine vascular remodeling by fetal-derived placental trophoblast cells. In many cases, PE can be diagnosed even in the absence of proteinuria if a patient has symptoms of end-organ dysfunction. GH, while considered less severe than PE, can progress to PE in most of the cases, making early and accurate differentiation between the two conditions clinically critical.

Further, PE can lead to severe consequences, such as placental ischemia, increased arterial resistance, maternal endothelial dysfunction, renal failure, eclamptic seizures, and maternal or fetal death. Although a precise cause of PE remains unknown, studies suggest that the origin of PE lies in abnormal placental development. In healthy pregnancies, a cytotrophoblast invasion of a uterine wall transforms uterine spiral arteries into a low-resistance vascular system, facilitating a transfer of oxygen and nutrients from a mother to a fetus. In PE, however, deficient cytotrophoblast invasion and insufficient transformation of the spiral arteries lead to placental ischemia and release of antiangiogenic factors. The antiangiogenic factors, such as sFlt-1 (soluble fts-like kinase 1) and Seng (soluble endoglin), inhibit blood vessel formation in the placenta, resulting in elevated blood pressure. GH, by contrast, does not involve the same degree of placental pathology as PE. GH is primarily characterized by elevated blood pressure arising from altered vascular tone and increased cardiac output, without significant endothelial dysfunction or antiangiogenic factor release. Despite this distinction, the hemodynamic changes in GH can still pose significant risks to both the mother and the fetus if left unmanaged.

In addition to anatomical changes, various genetic and environmental factors can also result in PE and GH. For instance, age, pregnancy interval, family history, obesity, comorbidities, ethnicity, and race are important contributing factors in occurrence of both the conditions. Women with a prior history of GH are also at an elevated risk of developing PE in subsequent pregnancies, underscoring a clinical overlap between the two conditions.

While the only known cure for PE is childbirth, many methods have been explored to reduce the morbidity and mortality, such as blood pressure control, incorporation of magnesium sulphate to prevent the eclamptic seizures, and delivery of a premature infant in severe cases. A management of GH similarly focuses on blood pressure monitoring and control, with close surveillance to detect any progression of GH to PE. In both the conditions, timely intervention is essential to avoid adverse maternal and fetal outcomes.

Over the years, a wide variety of clinical tools and diagnostic methods have been developed to detect PE and GH. While various ways to predict or detect the conditions have been investigated, a vast majority of the techniques require invasive methods, expensive equipment, or laboratory tests. Angiogenic markers have shown promise in identifying placental growth factors and soluble Fms-like tyrosine kinase-1, particularly for PE. However, high costs and limited assay availability restrict widespread use of such markers. For GH, a diagnosis currently relies predominantly on serial blood pressure measurements, which, while non-invasive, lack the specificity required to reliably distinguish GH from early-stage PE or to predict a disease progression.

Furthermore, arterial stiffness has been studied as a potential marker for both PE and GH. In a study performed on 69 normotensive and 54 preeclamptic pregnant women, Kaihura et al. detected a difference of 20% in a carotid to femoral median pulse wave velocity, with a further difference of 10% between a carotid artery and a radial artery, indicating increased maternal arterial stiffness in PE (Kaihura C et al. “Maternal arterial stiffness in pregnancies affected by preeclampsia.” Am J Physiol Heart Circ Physiol 2009 Aug;297(2):H759-H764). In a study by Arioz et al., 60 consecutive pregnant women in a third trimester were assessed using digital photoplethysmography and 24-hour ambulatory blood pressure monitoring, with 30 of the women being preeclamptic by standard criteria (Arioz DT et al. “Arterial stiffness and dipper/nondipper blood pressure status in women with preeclampsia.” Adv Ther. 2008 Sep;25(9) 925-934). The study calculated an arterial stiffness index (SI) from a digital volume pulse (DVP) obtained via pulse oximetry and monitored changes in the SI among the preeclamptic patients. However, the study failed to provide any output for identifying patients likely to develop PE or patients with asymptomatic PE.

Moreover, common clinical methods of detecting PE and GH include blood tests which detect prenatal cell-free DNA to identify genetic conditions such as Down's syndrome, Trisomy 21, and Trisomy 18, among other disorders. However, such methods are invasive, require a skilled professional to perform, and do not directly measure the hemodynamic or vascular changes specific to PE or GH. There therefore remains a need for a non-invasive, cost-effective, and accessible diagnostic tool capable of reliably detecting and differentiating PE and GH during pregnancy.

Hence there exists a need for a system and a method for predicting cardiovascular conditions in a subject during pregnancy which addresses at least the aforementioned problems.

### SUMMARY OF THE INVENTION

In an aspect, a system for predicting cardiovascular condition in a subject during pregnancy is disclosed. The system comprises a control unit. The control unit is configured to receive one or more physiological waveform data of the subject from the one or more sensors and a historical medical data of the subject. Further, the control unit is configured to derive a plurality of cardiovascular parameters based on an analysis of the received physiological waveform data of the subject. Moreover, the control unit is configured to process the derived cardiovascular parameters based on a trained predictive model, the predictive model being trained based on a pre-determined set of one or more cardiovascular conditions and the received historical medical data of the subject. Additionally, the control unit is configured to generate one or more cardiovascular risk scores based on the processing, thereby predicting the cardiovascular condition in the subject during pregnancy.

In an embodiment, the system comprises one or more sensors communicably coupled to the control unit. The one or more sensors are attached to the subject. The one or more sensors are configured to procure the one or more physiological waveform data of the subject. Further, the one or more sensors includes at least one of a photoplethysmography sensor configured to determine changes in blood volume in the subject and color of blood of the subject, a doppler ultrasonic sensor configured to determine blood flow in the subject, a pump and pressure sensor configured to determine a pulse pressure waveform of the subject and a thermistor sensor configured to determine a blood temperature of the subject.

In an embodiment, the physiological waveform data includes at least one of the blood volume, the blood flow, and the pulse pressure waveform.

In an embodiment, the plurality of cardiovascular parameters includes at least one of an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

In an embodiment, the historical medical data of the subject includes at least one of an age of the subject, a blood pressure at different gestational ages of the subject, a pre-disposition to hypertension of the subject, a family history of hypertension of the subject, a previous history of preeclampsia of the subject, a previous history of gestational hypertension of the subject, a presence of diabetes in the subject, a history of gestational diabetes in the subject, a medication regimen of the subject and a family history of the subject.

In an embodiment, the control unit is configured to combine a plurality of a cardiovascular health data of the subject, a cardiac output data of the subject and an arterial stiffness data of the subject. Further, the control unit generates a composite cardiovascular state representation of the subject, based on the combination. Furthermore, the control unit identifies a hemodynamic pattern indicative of a maladaptive cardiovascular adaptation during pregnancy, based on the generated composite cardiovascular state representation. Moreover, the control unit is configured to generate the one or more cardiovascular risk scores based on the identified hemodynamic pattern.

In an embodiment, the pre-determined set of one or more cardiovascular conditions correspond to at least one of a probability of developing a cardiovascular condition in the subject, a probability of developing Preeclampsia (PE) in the subject and the probability of developing Gestational Hypertension (GH) in the subject.

In an embodiment, the generated one or more cardiovascular risk scores is indicative of the probability of the cardiovascular condition in the subject. The one or more cardiovascular risk scores includes a first score. The first score is indicative of the probability of developing the cardiovascular condition in the subject. Further, the one or more cardiovascular risk scores includes a second score. The second score is indicative of the probability of developing the PE in the subject.

In an embodiment, the trained predictive model includes a multi-model predictive architecture configured to process the composite cardiovascular state representation using one or more trained predictive models operating simultaneously. The one or more trained predictive models includes a Bayesian model, a Tree-Based model, a Deep Learning Model and a Predictive and Discriminative Model. Further, the trained predictive model is configured to map the composite cardiovascular state representation, by using each of the one or more trained predictive models to a respective probability of the subject developing the cardiovascular condition. Furthermore, the trained predictive model is configured to merge the respective probability outputs of the one or more trained predictive models to generate a composite cardiovascular risk score for the subject.

In an embodiment, the system includes a display unit. The display unit is communicably coupled with the control unit. The display unit is configured to display at least one of the physiological waveform data, the plurality of cardiovascular parameters, the historical medical data of the subject, and the one or more cardiovascular risk scores.

In another aspect, a method for predicting a cardiovascular condition in a subject during pregnancy is disclosed. The method includes the step of receiving, one or more physiological waveform data of the subject, by a control unit. Further, the method includes the step of receiving a historical medical data of the subject by the control unit. Furthermore, the method includes the step of deriving a plurality of cardiovascular parameters based on an analysis of the received physiological waveform data of the subject. Thereafter, the method includes the step of processing the derived cardiovascular parameters based on a trained predictive model, the predictive model being trained based on a pre-determined set of one or more cardiovascular conditions and the received historical medical data of the subject by the control unit. Consequently, the method has the step of generating one or more cardiovascular risk scores based on the processing by the control unit, thereby predicting the cardiovascular condition in the subject during pregnancy.

In an embodiment, the physiological waveform data includes at least one of the blood volume, the blood flow, and the pulse pressure waveform.

In an embodiment, the plurality of cardiovascular parameters includes at least one of an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

In an embodiment, the historical medical data of the subject includes at least one of: an age of the subject, a blood pressure at different gestational ages of the subject, a pre-disposition to hypertension of the subject, a family history of hypertension of the subject, a previous history of preeclampsia of the subject, a previous history of gestational hypertension of the subject, a presence of diabetes in the subject, a history of gestational diabetes in the subject, a medication regimen of the subject and a family history of the subject.

In an embodiment, the method includes the step of combining, by the control unit, a plurality of a cardiovascular health data of the subject, a cardiac output data of the subject and an arterial stiffness data of the subject. Further, the method includes the step of generating, by the control unit, a composite cardiovascular state representation of the subject, based on the combination. Furthermore, the method includes the step of identifying, by the control unit, a hemodynamic pattern indicative of a maladaptive cardiovascular adaptation during pregnancy, based on the generated composite cardiovascular state representation. Moreover, the method includes the step of generating, by the control unit, the one or more cardiovascular risk scores based on the identified hemodynamic pattern.

In an embodiment, the pre-determined set of one or more cardiovascular conditions correspond to at least one of a probability of developing a cardiovascular condition in the subject, a probability of developing Preeclampsia (PE) in the subject and the probability of developing Gestational Hypertension (GH) in the subject.

In an embodiment, the generated one or more cardiovascular risk scores are indicative of the probability of the cardiovascular condition in the subject. The one or more cardiovascular risk scores includes a first score. The first score is indicative of the probability of developing the cardiovascular condition in the subject. Further, the one or more cardiovascular risk scores includes a second score. The second score is indicative of the probability of developing the PE in the subject.

In an embodiment, the method includes the step of processing, by the control unit, the composite cardiovascular state representation using one or more trained predictive models operating simultaneously. The one or more trained predictive models includes a Bayesian model, a Tree-Based model, a Deep Learning Model and a Predictive and Discriminative Model. Further, the method includes the step of mapping, by the control unit, the composite cardiovascular state representation, by each of the one or more trained predictive models to a respective probability of the subject developing the cardiovascular condition. Furthermore, the method includes the step of merging, by the control unit, the respective probability outputs of the one or more trained predictive models to generate a composite cardiovascular risk score for the subject.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1 illustrates a block diagram of a system for predicting a cardiovascular condition in a subject during pregnancy, in accordance with an embodiment of the present invention.

FIG. 2 illustrates a flow diagram of a method for predicting the cardiovascular condition in the subject during pregnancy, in accordance with an embodiment of the present invention.

### DETAILED DESCRIPTION OF THE INVENTION

Various features and embodiments of the present invention here will be discernible from the following further description thereof, set out hereunder.

The present invention relates to cardiovascular conditions. More particularly, the present invention relates to a system and a method for predicting a cardiovascular condition in a subject during pregnancy. The system has one or more sensors and a control unit. The one or more sensors are attached to the subject to procure one or more physiological waveform data of the subject. The control unit receives the one or more physiological waveform data and a historical medical data of the subject. The control unit derives a plurality of cardiovascular parameters based on an analysis of the received physiological waveform data and processes the derived cardiovascular parameters based on a trained predictive model. The predictive model is trained based on a pre-determined set of one or more cardiovascular conditions and the received historical medical data of the subject. The control unit generates one or more cardiovascular risk scores based on the processing, thereby predicting the cardiovascular condition in the subject during pregnancy. The procurement of the physiological waveform data through the one or more sensors avoids any invasive procedure, making the system particularly well-suited for use during pregnancy where minimizing procedural risk to the subject and the fetus is a primary clinical concern. The non-invasive nature of the system also supports patient compliance and comfort over extended monitoring periods.

Additionally, the control unit automatically derives the plurality of cardiovascular parameters from raw waveform data and processes those parameters through the trained predictive model without requiring manual clinical interpretation at each stage. This automation reduces the potential for human error, improves processing efficiency, and ensures that the cardiovascular risk scores are generated in a consistent and reproducible manner across different subjects and clinical settings.

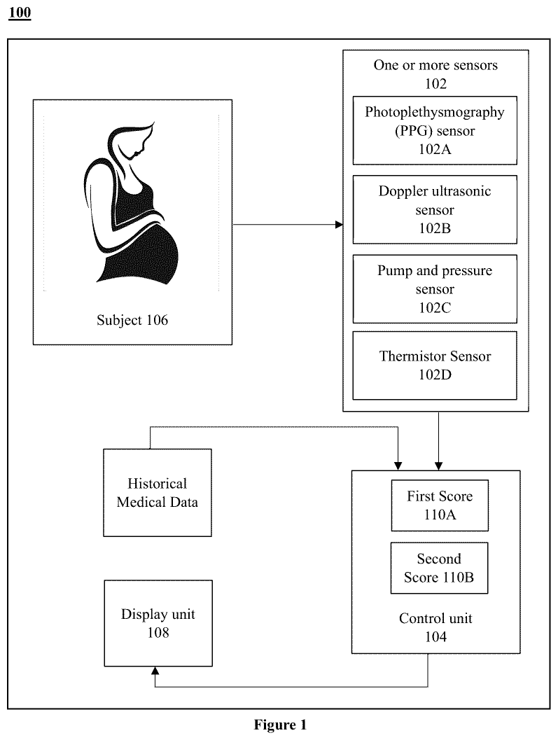

FIG. 1 illustrates a block diagram for a system 100 for predicting a cardiovascular condition in a subject 106 during pregnancy, in accordance with an embodiment of the present invention. Herein, the subject 106 refers to a pregnant woman undergoing cardiovascular monitoring during the course of her pregnancy. Given the significant physiological and hemodynamic changes that occur during pregnancy including increased cardiac output, altered blood pressure, and heightened risk of cardiovascular complications, the subject 106 represents a clinically vulnerable population requiring specialized and continuous monitoring. The system 106 is therefore configured to address the unique cardiovascular health needs of the pregnant women, with the objective of predicting and mitigating cardiovascular risk during this critical period.

Herein, the cardiovascular condition in the subject 106 during pregnancy refers to one or more pathological or clinically significant alterations in a cardiovascular and haemodynamic function of the subject 106 that arise, manifest, or are exacerbated during the gestational period, and which are associated with adverse maternal and foetal outcomes. In the present embodiment, the cardiovascular condition in the subject 106 during pregnancy relates to at least one of a Preeclampsia (PE) and a Gestational Hypertension (GH), without limiting the scope of the present invention.

In an embodiment, the PE as the cardiovascular condition in the subject 106 during pregnancy is characterised by the new onset of hypertension. As an example, the subject 106 is said to have the PE when a systolic blood pressure of 140 mmHg or greater, or a diastolic blood pressure of 90 mmHg or greater, measured on two or more occasions at least four hours apart, after the 20th week of gestation in the subject 106, in combination with one or more of proteinuria of 300 mg or greater in a 24-hour urine collection, a protein-to-creatinine ratio of 0.3 or greater, or a urine dipstick reading of 2+ or greater, or in the absence of proteinuria, in combination with new-onset thrombocytopaenia, renal insufficiency, impaired liver function, pulmonary oedema, or new-onset headache is unresponsive to medication in the subject 106.

In an embodiment, the GH as a cardiovascular condition in the subject 106 during pregnancy is characterised by the new onset of hypertension after the 20th week of gestation in the subject 106, in the absence of proteinuria or other systemic features of the PE. Further, the GH resolves within 12 weeks postpartum in the subject 106.

The system 100 comprises one or more sensors 102. The one or more sensors 102 are attached to the subject 106. The one or more sensors 102 are configured to procure one or more physiological waveform data of the subject 106. In an embodiment, the one or more sensors 102 comprise at least one of a photoplethysmography (PPG) sensor 102A, a doppler ultrasonic sensor 102B, a pump and pressure sensor 102C and a thermistor sensor 102D.

Further, the photoplethysmography (PPG) sensor 102A is configured to determine changes in blood volume in the subject 106 and color of blood of the subject 106. The PPG sensor 102A is an optical biosensing device that measures volumetric changes in blood flow within peripheral tissue by detecting variations in light absorption and reflection caused by a pulsatile nature of a cardiovascular system. The PPG sensor 102A operates by emitting light at one or more predetermined wavelengths, typically in the green (approximately 520-570 nm), red (approximately 620-750 nm), or near-infrared (approximately 750-1000 nm) spectrum. The one or more predetermined wavelengths are directed towards a tissue of the subject 106, which detects, by means of a photodetector, an intensity of light transmitted through or reflected from the tissue of the subject 106. In an embodiment, the PPG sensor 102A is configured to determine the color of the blood. Further, the control unit 104 may determine a dissolved oxygen in the blood using the PPG sensor 102a.

Furthermore, the doppler ultrasonic sensor 102B is configured to determine blood flow in the subject 106. The doppler ultrasonic sensor 102B operates on the principle of the doppler effect, wherein ultrasonic waves at a predetermined frequency are emitted towards a target blood vessel of the subject 106, and the frequency shift of the reflected ultrasonic waves is measured and analysed to determine the velocity and direction of blood flow within the said vessel. The doppler ultrasonic sensor 102B comprises a transducer element configured to both transmit and receive ultrasonic signals, thereby enabling continuous and non-invasive monitoring of the haemodynamic activity of the subject 106. The doppler ultrasonic sensor 102B is further configured to detect variations in blood flow velocity, including conditions of turbulent or restricted flow, which may be indicative of underlying vascular anomalies or physiological changes in the subject 106.

Moreover, the pump and pressure sensor 102C is configured to determine a pulse pressure waveform of the subject 106. The pump and pressure sensor 102C comprises a sphygmomanometer, which functions to measure and monitor the arterial blood pressure of the subject 106 by detecting pressure variations within an inflatable cuff applied to a limb of the subject 106. The sphygmomanometer operates by systematically inflating the cuff to a pressure exceeding the systolic blood pressure of the subject 106 and subsequently releasing the pressure in a controlled and gradual manner, thereby enabling the pump and pressure sensor 102C to capture and record the resulting pulse pressure waveform across a defined pressure range. The pulse pressure waveform thus obtained reflects the dynamic variation between the systolic and diastolic pressures, providing a continuous and accurate representation of the cardiovascular activity of the subject 106.

Additionally, the thermistor sensor 102D is configured to determine the blood temperature of the subject 106. As used herein, the thermistor sensor 102D is positioned on the skin surface of the subject 106 overlying superficial blood vessels, where they detect changes in skin temperature as a proxy for underlying blood temperature, offering a practical and patient-friendly alternative to invasive measurement.

As described herein, the term “physiological waveform data” in the context of the present invention relates to a time-series representation of one or more physiological parameters of the subject 106, generated from signals detected by the one or more sensors 102. The physiological waveform data comprises a plurality of data points, each data point corresponding to a measured value of the physiological parameter at a discrete point in time or over a defined time interval. The physiological waveform data reflects temporal variations in the physiological parameter across one or more physiological cycles of the subject. In the present embodiment, the physiological waveform data comprises at least one of: the blood volume, the blood flow, and the pulse pressure waveform. Therefore, the procurement of the physiological waveform data through the one or more sensors 102 avoids any invasive procedure, making the system 100 particularly well-suited for use during pregnancy where minimizing procedural risk to the subject 106 and the fetus is a primary clinical concern. The non-invasive nature of the system 100 also supports patient compliance and comfort over extended monitoring periods.

Further, the system 100 comprises a control unit 104. The control unit 104 is capable of executing machine executable instructions to perform the functions described herein. In some embodiments, a memory unit may be in communication with the control unit 104 is capable of storing machine executable instructions. Further, the control unit 104 is in communication with components such as the pre-processing module and the analytic module. In another embodiment, the control unit 104 is embodied as a multi-core processor, a single core processor, or a combination of one or more multi-core processors and one or more single core processors. For example, the control unit 104 is embodied as one or more of various processing devices, such as a coprocessor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like. In yet another embodiment, the control unit 104 is configured to execute hard-coded functionality.

In the present embodiment, the control unit 104 is communicably coupled with the one or more sensors 102. The control unit 104 is configured to receive the one or more physiological waveform data of the subject 106 from the one or more sensors 102. As an example, the control unit 104 receives the one or more physiological waveform data of the subject 106 directly or indirectly from the one or more sensors 102. For instance, the one or more physiological waveform data can be acquired from a data source present in a cloud using an Application Programming Interface (API). The control unit 104 is also configured to receive a historical medical data of the subject 106.

In an embodiment, the historical medical data of the subject comprises at least one of an age of the subject 106, a blood pressure at different gestational ages of the subject 106, a pre-disposition to hypertension of the subject 106, a family history of hypertension of the subject 106, a previous history of preeclampsia of the subject 106, a previous history of gestational hypertension of the subject 106, a presence of diabetes in the subject 106, a history of gestational diabetes in the subject 106, a medication regimen of the subject 106 and a family history of the subject 106.

Herein, the term “blood pressure at different gestational ages of the subject 106” in the context of the present invention refers to the recorded systolic and diastolic arterial blood pressure measurements of the subject 106 obtained at one or more discrete time points across the gestational period of the subject 106. In an embodiment, the gestational period is divided into a plurality of gestational intervals, comprising a first trimester (approximately 1 to 13 weeks of gestation), a second trimester (approximately 14 to 26 weeks of gestation), and a third trimester (approximately 27 to 40 weeks of gestation), and the blood pressure measurements of the subject 106 are recorded at one or more time points within each of the said gestational intervals.

Herein, the term “pre-disposition to hypertension of the subject 106” in the context of the present invention refers to an inherent or acquired susceptibility of the subject 106 to develop elevated arterial blood pressure, either prior to or during the gestational period, arising from one or more constitutional, physiological, or lifestyle-related risk factors such as chronic hypertension in the subject 106, a body mass index (BMI) of the subject 106 exceeding a clinically established threshold, a history of renal or endocrine disorders in the subject 106, a sedentary lifestyle or elevated stress levels of the subject 106, and the presence of comorbid conditions such as diabetes or obesity in the subject 106.

Herein, the term “family history of hypertension of the subject 106” in the context of the present invention refers to a documented occurrence of hypertension or hypertension-related cardiovascular conditions in one or more biological relatives of the subject 106, including but not limited to first-degree relatives such as a biological parent or sibling of the subject 106, and second-degree relatives such as a grandparent, aunt, or uncle of the subject 106.

Herein, the term “previous history of Preeclampsia of the subject 106” refers to a prior documented diagnosis of the PE in the subject 106 during one or more preceding gestational periods, wherein PE was clinically established based on the onset of hypertension and proteinuria or other end-organ dysfunction manifesting after the 20th week of gestation in the subject 106.

Herein, the term “a presence of diabetes in the subject 106” in the context of the present invention refers to pre-existing diabetes in the subject 106 refers to a diagnosis of Type 1 Diabetes Mellitus (T1DM) or Type 2 Diabetes Mellitus (T2DM) established prior to the current gestational period in the subject 106, wherein T1DM is characterised by absolute insulin deficiency resulting from autoimmune destruction of pancreatic beta cells in the subject 106, and T2DM is characterised by progressive insulin resistance and relative insulin deficiency in the subject 106.

Herein, the term “a history of gestational diabetes in the subject 106” in the context of present invention refers to a form of diabetes in the subject 106 first recognised or diagnosed during the gestational period in the subject 106.

Herein, the term “medication regimen of the subject 106” in the context of the present invention refers to a complete and structured record of all pharmacological agents, therapeutic drugs, supplements, and prophylactic medications prescribed to, administered to, or consumed by the subject 106 which also includes a drug name, dosage, frequency, and route of administration, as recorded prior to or during the gestational period of the subject 106.

Herein, the term “family history of the subject” in the context of the present invention refers to the documented record of medical conditions, diseases, and disorders previously diagnosed in or experienced by the one or more biological relatives of the subject 106.

Further, the control unit 104 is configured to derive a plurality of cardiovascular parameters based on an analysis of the received physiological waveform data of the subject 106. In an embodiment, the derivation of the plurality of cardiovascular parameters is performed by the control unit 104 by analysing the physiological waveform data of the subject 106 through a series of signal processing operations. In an embodiment, the signal processing operations include signal conditioning, wherein the raw physiological waveform data received from the one or more wearable sensors 102 is amplified and normalised to a predetermined amplitude range so as to obtain a conditioned physiological waveform signal of the subject 106.

In an embodiment, the signal processing operations further include filtering of the conditioned physiological waveform signal of the subject 106, wherein one or more of a low-pass filter, a high-pass filter, a band-pass filter, and a notch filter are applied to the conditioned physiological waveform signal so as to remove one or more of high-frequency noise, baseline wander, motion artefacts, and powerline interference from the conditioned physiological waveform signal of the subject 106, thereby obtaining a filtered physiological waveform signal of the subject 106.

In an embodiment, the plurality of cardiovascular parameters comprises at least one of an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

Herein, the “aortic diastolic blood pressure” in the context of the present invention refers to a minimum pressure within an aorta of a heart of the subject 106 at an end of a cardiac relaxation phase. The aortic diastolic blood pressure is derived by the control unit 104 from the blood pressure waveform data obtained through the pump and pressure sensor 102C.

Herein, the “aortic systolic blood pressure” in the context of the present invention refers to a peak pressure within the aorta of the subject 106 during a ventricular ejection phase of the cardiac cycle. The aortic systolic blood pressure is derived by the control unit 104 from a peripheral blood pressure waveform obtained through the pump and pressure sensor 102C, wherein a generalised transfer function is applied to the peripheral pressure waveform so as to reconstruct a central aortic pressure waveform, and a maximum amplitude value of each cardiac cycle in the reconstructed central aortic pressure waveform is identified as the aortic systolic blood pressure of the subject 106.

Herein, the “aortic pulse pressure” in the context of the present invention refers to a difference between the aortic systolic blood pressure and the aortic diastolic blood pressure of the subject 106. The aortic pulse pressure serves as an indicator of an arterial stiffness and a cardiovascular load. The aortic pulse pressure is derived by the control unit 104 by computing an arithmetic difference between the derived aortic systolic blood pressure and the derived aortic diastolic blood pressure of the subject 106 from the reconstructed central aortic pressure waveform, wherein a higher aortic pulse pressure is indicative of increased arterial stiffness in the subject 106.

Herein, the “augmentation pressure” in the context of the present invention refers to an additional pressure imposed on a left ventricle of the subject 106 as a result of an early return of reflected pressure waves from a peripheral vasculature during a systolic phase of the cardiac cycle. The augmentation pressure is derived by the control unit 104 from the reconstructed central aortic pressure waveform by computing a difference between the second systolic peak and an inflection point of the central aortic pressure waveform of the subject 106, wherein the inflection point is identified by applying first and second derivative analyses to the central aortic pressure waveform.

Herein, the “augmentation index” in the context of the present invention refers to a ratio of the augmentation pressure to the aortic pulse pressure of the subject 106. The augmentation index is expressed as a percentage and is utilised as a measure of the arterial stiffness and wave reflection in the subject 106. The augmentation index is derived by the control unit 104 from the reconstructed central aortic pressure waveform by identifying the inflection point in the waveform that corresponds to the arrival of the reflected pressure wave.

Herein, the “cardiac output” in the context of the present invention refers to a total volume of blood ejected by the left ventricle of the subject 106 per unit time. The cardiac output is utilised as a primary indicator of overall cardiac performance. The cardiac output is derived by the control unit 104 by computing the product of the pulse pressure stroke volume and a heart rate of the subject 106, wherein the heart rate is derived from an inter-beat interval of the physiological waveform data obtained via the PPG sensor 102A of the subject 106.

Herein, the “cardiac index” in the context of the present invention refers to the cardiac output of the subject 106 normalised to a body surface area of the subject 106 and is utilised as a size-independent measure of the cardiac performance. The cardiac index is derived by the control unit 104 by computing a ratio of the derived cardiac output of the subject 106 to the body surface area of the subject 106, wherein the body surface area is estimated from a height and a weight of the subject 106.

Herein, the “end systolic pressure” in the context of the present invention refers to the aortic pressure of the subject 106 at a moment of aortic valve closure which represents an afterload experienced by the left ventricle at the end of the systolic phase of the cardiac cycle. The end systolic pressure is derived by the control unit 104 from the reconstructed central aortic pressure waveform by identifying the pressure value at a dicrotic notch of the reconstructed central aortic pressure waveform. In an embodiment, the dicrotic notch corresponds to a closure of the aortic valve.

Herein, the “end systolic pressure index” in the context of the present invention refers to the end systolic pressure of the subject 106 normalised to the body surface area of the subject 106. The end systolic pressure index is utilised as a size-independent measure of a left ventricular afterload. Further, the end systolic pressure index is derived by the control unit 104 by computing the ratio of the derived end systolic pressure of the subject 106 to the body surface area of the subject 106.

Herein, the “mean arterial pressure” in the context of the present invention refers to an average arterial pressure throughout a single cardiac cycle of the subject 106. The mean arterial pressure represents a perfusion pressure driving blood flow to peripheral tissues and organs of the subject 106. The mean arterial pressure is derived by the control unit 104 from the blood pressure waveform data obtained via the pump and pressure sensor 102C. In an embodiment, the mean arterial pressure is computed as a time-weighted mean of the continuous blood pressure waveform over a single cardiac cycle or approximated as a sum of the diastolic blood pressure and a one-third of the pulse pressure of the subject 106.

Herein, the “pulse pressure stroke volume” in the context of the present invention refers to a volume of blood ejected by the left ventricle of the subject 106 per cardiac cycle. The pulse pressure stroke volume is estimated from the pulse pressure of the subject 106 and compliance characteristics of the arterial system of the subject 106. The pulse pressure stroke volume is derived by the control unit 104 from the blood pressure waveform data obtained via the pump and pressure sensor 102C. In an embodiment, the pulse pressure stroke volume is computed as a product of the aortic pulse pressure and a total arterial compliance of the subject 106 which may be estimated using an established pulse contour method applied to the blood pressure waveform.

Herein, the term “subendocardial viability ratio” in the context of the present invention refers to a ratio of a diastolic pressure-time index to a systolic pressure-time index of the subject 106. The subendocardial viability ratio is utilised as an indicator of a myocardial oxygen supply relative to a myocardial oxygen demand, particularly with respect to a subendocardial layer of the left ventricle of the subject 106. The subendocardial viability ratio is derived by the control unit 104 from the reconstructed central aortic pressure waveform by computing an area under the diastolic portion of the waveform that represents myocardial perfusion.

Herein, the “total peripheral resistance” in the context of the present invention refers to a cumulative resistance offered by the systemic vasculature of the subject 106 to a blood flow. The toral peripheral resistance is utilised as an indicator of a vascular tone and an afterload of the subject 106. The total peripheral resistance is derived by the control unit 104 by computing a ratio of the mean arterial pressure of the subject 106 to the cardiac output of the subject 106.

Based on the plurality of cardiovascular parameters derived by the control unit 104, the control unit 104 then determines one or more of: a cardiovascular health data of the subject 106, a cardiac output data of the subject 106 and an arterial stiffness data of the subject 106.

Herein, the term “cardiovascular health data” in the context of the present invention refers to a dataset determined by the control unit 104, representative of the haemodynamic and cardiovascular state of the subject 106 during the gestational period. The cardiovascular health data is determined based on one or more of the following cardiovascular parameters derived from the physiological waveform data of the subject 106: the mean arterial pressure, the pulse pressure, the heart rate, the subendocardial viability ratio, the aortic systolic blood pressure, and the aortic diastolic blood pressure of the subject 106. In an embodiment, a plurality of cardiovascular health data is determined by the control unit 104 at one or more discrete time points across the gestational period of the subject 106, comprising measurements recorded within the first trimester, the second trimester, and the third trimester of the subject 106.

Herein, the term “cardiac output data” in the context of the present invention refers to a dataset determined by the control unit 104, representative of the pumping performance of the heart of the subject 106. The cardiac output data is determined based on one or more of the following parameters: the stroke volume, the cardiac output, and the cardiac index of the subject 106. In an embodiment, the control unit 104 determines a cardiac output based on at least one of the stroke volume, the cardiac output, and the cardiac index.

The term “arterial stiffness data” in the context of the present invention refers to a dataset determined by the control unit 104, representative of the compliance and rigidity of the arterial walls of the subject 106. The arterial stiffness data is determined based on one or more of the following cardiovascular parameters derived from the reconstructed central aortic pressure waveform of the subject 106: the aortic pulse pressure and the augmentation index of the subject 106. In an embodiment, the control unit 104 determines an arterial stiffness data based on at least one of the aortic pulse pressure and the augmentation index.

In an embodiment, the control unit 104 is configured to combine the plurality of the cardiovascular health data of the subject 106, the cardiac output data of the subject 106 and the arterial stiffness data of the subject 106. Thereafter, the control unit 104 generates a composite cardiovascular state representation of the subject 106, based on the combination. Therefore, the control unit 104 generates the composite cardiovascular state representation of the subject 106 by combining three distinct categories of physiological data such as the plurality of cardiovascular health data, the cardiac output data, and the arterial stiffness data into a unified representation that reflects the overall cardiovascular condition of the subject 106 at any given point during monitoring. This integrated representation ensures that no single parameter is assessed in isolation, rather the interrelationship between cardiac function, vascular compliance, and broader cardiovascular health indicators is collectively analyzed to produce a comprehensive and holistic picture of the cardiovascular state of the subject 106. By consolidating these multiple data streams into a single composite representation, the system 100 is able to capture the full complexity of the cardiovascular physiology of the subject 106, thereby enhancing the accuracy and clinical reliability of any subsequent risk assessment or predictive analysis performed by the system 100.

Consequently, the control unit 104 is configured to identify a hemodynamic pattern indicative of a maladaptive cardiovascular adaptation during pregnancy, based on the generated composite cardiovascular state representation. Specifically, based on the generated composite cardiovascular state representation, the system 100 analyses the underlying haemodynamic patterns of the subject 106 to identify any deviation from the expected and physiologically normal cardiovascular adaptations that typically occur during pregnancy. Herein, the “maladaptive cardiovascular adaptation” refers to a condition wherein the cardiovascular system of the subject 106 fails to appropriately adjust to the increased physiological demands of pregnancy such as the required increase in cardiac output, reduction in vascular resistance, and maintenance of adequate uteroplacental perfusion, thereby giving rise to abnormal haemodynamic patterns that may signal the onset of serious pregnancy-related cardiovascular complications. By evaluating the composite cardiovascular state representation, which integrates cardiovascular health data, cardiac output data, and arterial stiffness data, the system 100 is capable of detecting such maladaptive patterns with a high degree of clinical precision, enabling early identification of conditions such as the PE, GE, or other cardiovascular disorders that may pose a significant risk to both the subject 106 and the foetus if left undetected or unmanaged

For this, the control unit 104 is configured to process the derived cardiovascular parameters based on a trained predictive model. The predictive model is trained based on a pre-determined set of one or more cardiovascular conditions and the received historical medical data of the subject 106. In an embodiment, the pre-determined set of one or more cardiovascular conditions correspond to at least one of a probability of developing a cardiovascular condition in the subject 106, a probability of developing the PE in the subject 106 and the probability of developing the GH in the subject 106. Based on processing the derived cardiovascular parameters, the control unit 104 generates one or more cardiovascular risk scores 110 based on the processing, thereby predicting the cardiovascular condition in the subject 106 during pregnancy,

In an embodiment, the trained predictive model comprises a multi-model predictive architecture configured to process the composite cardiovascular state representation using one or more trained predictive models operating simultaneously. Herein, the one or more trained predictive models comprises a Bayesian model, a Tree-Based model, a Deep Learning Model and a Predictive and Discriminative Model.

In an embodiment, the Bayesian model is configured to integrate outputs from a plurality of constituent models, wherein each constituent model of the plurality of constituent models is configured to capture one or more distinct aspects of the cardiovascular condition in the subject 106 during pregnancy. The Bayesian model is configured to reduce structural uncertainty arising from the nonlinear and temporally evolving interactions between pathophysiological, vascular, metabolic, and environmental factors associated with the cardiovascular condition in the subject 106 during pregnancy.

In an embodiment, the trained predictive model comprises a Bayesian ensemble model configured to integrate outputs from a plurality of constituent models. Each constituent model of the plurality of constituent models is configured to capture one or more distinct aspects of the cardiovascular condition in the subject 106 during pregnancy. The Bayesian ensemble model is configured to reduce structural uncertainty arising from the nonlinear and temporally evolving interactions between pathophysiological, vascular, metabolic, and environmental factors associated with the cardiovascular condition in the subject 106 during pregnancy.

In an embodiment, the Tree-Based model is configured to evaluate one or more of the following features derived from the composite cardiovascular state representation at each decision node: the mean arterial pressure, the augmentation index, the aortic pulse pressure, the total peripheral resistance, the cardiac output, and the subendocardial viability ratio of the subject 106. The Tree-Based model is further configured to assign an importance score to each of the said features, thereby enabling the control unit 104 to identify the cardiovascular parameters most predictive of the cardiovascular condition in the subject 106.

In an embodiment, the Deep Learning model comprises one or more of a recurrent neural network (RNN), a long short-term memory (LSTM) network, a convolutional neural network (CNN), and a transformer-based neural network. In an embodiment wherein a recurrent architecture is employed, the Deep Learning model is configured to process the composite cardiovascular state representation as a temporal sequence of haemodynamic measurements recorded across one or more gestational intervals of the subject 106 comprising the first trimester, the second trimester, and the third trimester, thereby enabling the model to learn longitudinal haemodynamic trajectories predictive of the PE or GH in the subject 106.

In an embodiment, the Deep Learning model is trained on a training dataset comprising physiological waveform data, derived cardiovascular parameters, and confirmed clinical outcomes of a plurality of pregnant subjects, wherein the model parameters are optimised through backpropagation using a binary cross-entropy loss function computed against the ground truth clinical labels of the PE and GH.

In an embodiment, the predictive function of the Predictive and Discriminative model comprises one or more of a logistic regression classifier, a support vector machine (SVM), and a linear discriminant analysis (LDA) model, each trained to map the composite cardiovascular state representation to a respective probability of the subject 106 developing the cardiovascular condition. The discriminative function of the Predictive and Discriminative model is configured to compute a discriminant score representative of the degree of separation between the PE and GH classes in the composite cardiovascular state representation space of the subject 106.

In an embodiment, the Predictive and Discriminative model is further configured to incorporate the historical medical data of the subject 106 such the previous history of PE, the pre-disposition to hypertension, and the medication regimen of the subject 106 as auxiliary inputs to the discriminative function, thereby improving the specificity of the discrimination between the PE and GH in the subject 106.

Further, the trained predictive model is configured map the composite cardiovascular state representation, by each of the one or more trained predictive models to a respective probability of the subject developing the cardiovascular condition. In an embodiment, the control unit 104 is configured to map the composite cardiovascular state representation of the subject 106, by each of the one or more trained predictive models operating simultaneously, to a respective probability of the subject 106 developing the cardiovascular condition during pregnancy. The composite cardiovascular state representation comprising the cardiovascular health data, the cardiac output data, and the arterial stiffness data of the subject 106 is provided as a common input to each of the Bayesian model, the Tree-Based model, the Deep Learning model, and the Predictive and Discriminative model. Each trained predictive model independently processes the composite cardiovascular state representation in accordance with its respective architecture and trained parameters, and outputs a respective probability value in the range of 0 (zero) to 1 (one), wherein a value closer to 1 (one) is indicative of a higher likelihood of the subject 106 developing the cardiovascular condition during the pregnancy.

In an embodiment, the control unit 104 is configured to merge the respective probability outputs of the Bayesian model, the Tree-Based model, the Deep Learning model, and the Predictive and Discriminative model to generate the composite cardiovascular risk score for the subject 106. The merging is performed by computing a weighted aggregation of the respective probability outputs of each of the one or more trained predictive models, wherein a respective weight is assigned to each trained predictive model based on the relative predictive performance of that model on a validation dataset comprising confirmed clinical outcomes of the PE and GH in a plurality of pregnant subjects, such that trained predictive models demonstrating higher predictive accuracy on the validation dataset are assigned a proportionally greater contribution to the composite cardiovascular risk score of the subject 106.

In an embodiment, the control unit 104 is configured to generate the one or more cardiovascular risk scores 110 based on the identified haemodynamic pattern of the subject 106. In an embodiment, each of the one or more cardiovascular risk scores 110 is a discrete numerical value on a predefined risk scale, wherein a higher cardiovascular risk score is indicative of a greater magnitude of haemodynamic deviation and a correspondingly elevated likelihood of the subject 106 developing the PE or GH during the gestational period.

In an embodiment, the generated one or more cardiovascular risk scores 110 are indicative of the probability of the cardiovascular condition in the subject. The one or more cardiovascular risk scores 110 comprises a first score 110A and a second score 110B. The first score 110A is indicative of the probability of developing the cardiovascular condition in the subject 106. In an embodiment, the first score 110A is indicative of the probability of developing the cardiovascular condition such as the PE or the GH in the subject 106. The second score 110B is indicative of the probability of developing the PE in the subject 106. Herein, if the first score 110A indicates the probability of developing either the PE or the GH in the subject 106, then the second score 110B indicates the probability of developing the PE in the subject 106. If there is no probability of developing the PE in the subject 106, then the second score 110B indicates the probability of developing the GH in the subject 106.

In an embodiment, each of the first score 110A and the second score 110B is derived from the probability outputs generated by the one or more trained predictive models upon processing the cardiac output data, the arterial stiffness data, and the cardiovascular health data of the subject 106. In an embodiment, the probability outputs generated by each of the one or more trained predictive models are normalised to a common scale, and a weighted aggregation of the normalised probability outputs is performed, wherein each trained predictive model is assigned a predefined weight reflective of its relative predictive accuracy and clinical relevance to the specific cardiovascular condition being assessed. The first score 110A is indicative of the probability of developing the cardiovascular condition in the subject 106 and is computed as a weighted aggregate of the normalised probability outputs of the one or more trained predictive models corresponding to the overall cardiovascular risk profile of the subject 106.

In an embodiment, the second score 110B is indicative of the probability of developing the PE in the subject 106, and is computed as a weighted aggregate of the normalised probability outputs of the one or more trained predictive models specifically associated with haemodynamic markers and physiological parameters predictive of PE, thereby enabling the healthcare provider to undertake a more targeted and clinically informed approach to the monitoring and management of the subject 106 throughout the gestational period.

In an example, consider a scenario wherein the trained predictive models described hereinabove, namely the Bayesian model, the Tree-Based model, the Deep Learning model, and the Predictive and Discriminative model, are employed to assess the cardiovascular risk of the subject 106. Upon processing the cardiac output data, the arterial stiffness data, and the cardiovascular health data of the subject 106, the Bayesian model, the Tree-Based model, the Deep Learning model, and the Predictive and Discriminative model generate normalised probability outputs of 0.74, 0.81, 0.88, and 0.79, respectively, with predefined weights of 0.25, 0.25, 0.30, and 0.20 assigned to each model based on their relative predictive accuracy and clinical relevance. The first score 110A is thereby computed as a weighted aggregate of the said normalised probability outputs, wherein (0.74×0.25)+(0.81×0.25)+(0.88×0.30)+(0.79×0.20)=0.8075, indicating a high probability of developing the cardiovascular condition in the subject 106 and prompting the healthcare provider to initiate appropriate clinical intervention.

In another example, consider a scenario wherein the trained predictive models described hereinabove are employed progressively to assess the cardiovascular risk of the subject 106, wherein each successive model serves as a more advanced calibration layer upon the probability output generated by the preceding model, and wherein a normalised probability output falling within the range of 0 to 0.5 indicates a likelihood of the GH and a normalised probability output falling within the range of 0.5 five to 1 indicates a likelihood of the PE.

In a first instance, the Bayesian model, being the least complex of the four models, processes the cardiac output data, the arterial stiffness data, and the cardiovascular health data of the subject 106 and generates an initial normalised probability output of 0.55, placing the subject 106 within the PE range, albeit at a low confidence level proximate to the GH-PE boundary. Since the said initial probability output, whilst falling within the PE range, remains proximate to the classification boundary and thereby warrants further calibration, the system 100 escalates the assessment to the Tree-Based model, which further processes the said data alongside the probability output of the Bayesian model as an additional input feature, and generates a refined normalised probability output of 0.63, reflecting a greater degree of separation from the GH-PE boundary and a more confident classification of the subject 106 within the PE range.

Further, the refined probability output indicates the PE but continues to warrant validation given the clinical significance of the condition. Therefore, the system 100 escalates the assessment to the Deep Learning model, which, by virtue of its capacity to identify complex non-linear patterns across the said data, further calibrates the probability output and generates a more precise normalised probability output of 0.74, reinforcing the classification of the subject 106 within the PE range with a higher degree of predictive confidence. Since the said more precise probability output further strengthens the PE classification, the system 100 escalates the assessment to the Predictive and Discriminative model that integrates both generative and discriminative learning paradigms to perform a final calibration of the probability output and generates a conclusive normalised probability output of 0.82. The second score 110B is thereby determined as 0.82, conclusively classifying the subject 106 within the PE range and indicating a high probability of PE, thereby prompting the healthcare provider to initiate appropriate clinical intervention.

In an embodiment, the system 100 comprises a display unit 108. The display unit 108 is communicably coupled with the control unit 104. Further, the display unit 108 is configured to display at least one of: the physiological waveform data, the plurality of cardiovascular parameters, the historical medical data of the subject 106, and the one or more cardiovascular risk scores. In an embodiment, the display unit 108 maybe a user device such as a computer, a mobile device, and the like.

Therefore, the system 100 provides a non-invasive, continuous, and real-time monitoring solution that simultaneously captures cardiovascular health data, cardiac output data, and arterial stiffness data, thereby eliminating the need for multiple discrete diagnostic tools and significantly reducing the clinical burden on both the subject 106 and the healthcare provider. By generating the composite cardiovascular state representation and leveraging the trained predictive model to identify maladaptive haemodynamic patterns, the system 100 enables early and accurate detection of pregnancy-related cardiovascular complications such as the PE and the GH at a stage where timely clinical intervention can meaningfully alter patient outcomes. The integration of multiple physiological data streams into a single unified platform ensures that the cardiovascular condition of the subject 106 is assessed holistically rather than in isolation, thereby enhancing diagnostic accuracy, reducing the risk of missed or delayed diagnoses, and improving the overall reliability of risk stratification during pregnancy. Collectively, these capabilities position the system 100 as a comprehensive, intelligent, and clinically actionable cardiovascular monitoring solution that advances the standard of maternal healthcare by combining continuous physiological surveillance with predictive analytical intelligence.

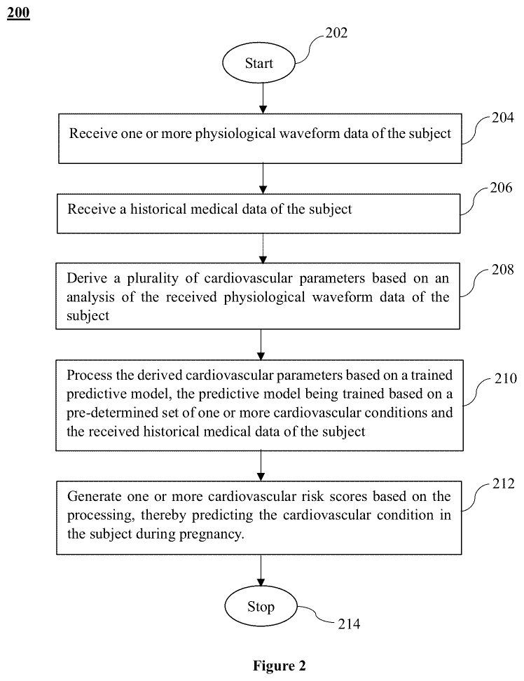

FIG. 2 illustrates a flow diagram of a method 200 for predicting the cardiovascular condition in the subject 106 during pregnancy, in accordance with an embodiment of the present invention. The method 200 is performed on the system 100 for predicting the cardiovascular condition in the subject 106 during pregnancy, as described hereinabove. The method 200 may be realized through executable instructions configured to be executed by the system's circuitry and components such as the control unit 104. The system 100 for predicting the cardiovascular condition in the subject 106 during pregnancy and the method 200 collectively enable the functionalities described herein to achieve the intended technical effect. The method 200 for predicting the cardiovascular condition in the subject 106 during pregnancy starts at step 202. The method 200 then proceeds to step 204.

At step 204, the method 200 comprises the step of receiving, by the control unit 104, the one or more physiological waveform data of the subject 106 from the one or more sensors 102. In an embodiment, the control unit 104 is configured to receive the physiological waveform data of the subject 106 from a server (not shown). In an embodiment and as described hereinabove, the one or more sensors 102 comprise at least one of the photoplethysmography (PPG) sensor 102A, the doppler ultrasonic sensor 102B, and the pump and pressure sensor 102C. The photoplethysmography (PPG) sensor 102A is configured to determine changes in blood volume in the subject 106. The doppler ultrasonic sensor 102B is configured to determine blood flow in the subject 106. The pump and pressure sensor 102C is configured to determine a pulse pressure waveform of the subject 106.

Herein, the physiological waveform data is generated based on signals from the one or more sensors 102. In an embodiment, the physiological waveform data comprises at least one of a blood volume, a blood flow, and a pulse pressure waveform. Thereafter, the method 200 proceeds to step 206.

At step 206, the method 200 comprises the step of receiving, by the control unit 104, a historical medical data of the subject 106. In an embodiment, the historical medical data of the subject comprises at least one of an age of the subject 106, a blood pressure at different gestational ages of the subject 106, a pre-disposition to hypertension of the subject 106, a family history of hypertension of the subject 106, a previous history of preeclampsia of the subject 106, a previous history of gestational hypertension of the subject 106, a presence of diabetes in the subject 106, a history of gestational diabetes in the subject 106, a medication regimen of the subject 106 and a family history of the subject 106. Thereafter, the method 200 proceeds to step 208.

At step 208, the method 200 comprises the step of deriving, by the control unit 104, a plurality of cardiovascular parameters based on an analysis of the received physiological waveform data of the subject 106. In an embodiment, the derivation of the plurality of cardiovascular parameters is performed by the control unit 104 by analysing the physiological waveform data of the subject 106 through a series of signal processing operations. In an embodiment, the signal processing operations include signal conditioning, wherein the raw physiological waveform data received from the one or more wearable sensors 102 is amplified and normalised to a predetermined amplitude range so as to obtain a conditioned physiological waveform signal of the subject 106.

In an embodiment, the signal processing operations further include filtering of the conditioned physiological waveform signal of the subject 106, wherein one or more of a low-pass filter, a high-pass filter, a band-pass filter, and a notch filter are applied to the conditioned physiological waveform signal so as to remove one or more of high-frequency noise, baseline wander, motion artefacts, and powerline interference from the conditioned physiological waveform signal of the subject 106, thereby obtaining a filtered physiological waveform signal of the subject 106.

In an embodiment, the plurality of cardiovascular parameters comprises at least one of an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

At step 210, the method 200 comprises the step of processing, by the control unit 104, the derived cardiovascular parameters based on a trained predictive model. Herein, the predictive model is trained based on a pre-determined set of one or more cardiovascular conditions and the received historical medical data of the subject 106. In an embodiment, the pre-determined set of one or more cardiovascular conditions correspond to at least one of a probability of developing a cardiovascular condition in the subject 106, a probability of developing the PE in the subject 106 and the probability of developing the GH in the subject 106.

Further, based on the derived plurality of cardiovascular parameters, the control unit 104 is configured to determine one or more of a plurality of a cardiovascular health data of the subject 106, a cardiac output data of the subject 106 and an arterial stiffness data of the subject 106, as described hereinabove.

In an embodiment, the method 200 comprises the step of combining, by the control unit 104, the plurality of a cardiovascular health data of the subject 106, the cardiac output data of the subject 106 and the arterial stiffness data of the subject 106. Further, the method 200 comprises the step of generating, by the control unit 104, a composite cardiovascular state representation of the subject 106, based on the combination. Furthermore, the method 200 comprises the step of identifying, by the control unit 104, a hemodynamic pattern indicative of a maladaptive cardiovascular adaptation during pregnancy, based on the generated composite cardiovascular state representation.

In an embodiment, the trained predictive model comprises a multi-model predictive architecture configured to process the composite cardiovascular state representation using one or more trained predictive models operating simultaneously, the one or more trained predictive models comprising: a Bayesian model, a Tree-Based model, a Deep Learning Model and a Predictive and Discriminative Model.

In an embodiment, the method 200 has the step of processing, by the control unit 106, the composite cardiovascular state representation using the one or more trained predictive models operating simultaneously. The one or more trained predictive models comprising a Bayesian model, a Tree-Based model, a Deep Learning Model and a Predictive and Discriminative Model.

In an embodiment, the Bayesian model configured to integrate outputs from a plurality of constituent models, wherein each constituent model of the plurality of constituent models is configured to capture one or more distinct aspects of the cardiovascular condition in the subject 106 during pregnancy.

In an embodiment, the Tree-Based model is configured to evaluate one or more of the following features derived from the composite cardiovascular state representation at each decision node: the mean arterial pressure, the augmentation index, the aortic pulse pressure, the total peripheral resistance, the cardiac output, and the subendocardial viability ratio of the subject 106.

In an embodiment, the Deep Learning model comprises one or more of a recurrent neural network (RNN), a long short-term memory (LSTM) network, a convolutional neural network (CNN), and a transformer-based neural network.

In an embodiment, the Deep Learning model is trained on a training dataset comprising physiological waveform data, derived cardiovascular parameters, and confirmed clinical outcomes of a plurality of pregnant subjects, wherein the model parameters are optimised through backpropagation using a binary cross-entropy loss function computed against the ground truth clinical labels of the PE and the GH.

In an embodiment, the predictive function of the Predictive and Discriminative model comprises one or more of a logistic regression classifier, a support vector machine (SVM), and a linear discriminant analysis (LDA) model, each trained to map the composite cardiovascular state representation to a respective probability of the subject 106 developing the cardiovascular condition.

In an embodiment, the method 200 comprises the step of mapping, by the control unit 106, the composite cardiovascular state representation, by each of the one or more trained predictive models to a respective probability of the subject 106 developing the cardiovascular condition. The method 200 then proceeds to step 212.

At step 212, the method 200 comprises the step of generating, by the control unit 104, one or more cardiovascular risk scores 110 based on the processing, thereby predicting the cardiovascular condition in the subject 106 during pregnancy. In an embodiment, the method 200 comprises the step of generating, by the control unit 104, the one or more cardiovascular risk scores 110 based on the identified hemodynamic pattern. In an embodiment, the method 200 comprises the step of merging, by the control unit 106, the respective probability outputs of the one or more trained predictive models to generate one or more cardiovascular risk scores 110 based on the processing. Specifically, the control unit 104 is configured to generate a composite cardiovascular risk score 110 for the subject 106.

In an embodiment, the generated one or more cardiovascular risk scores are indicative of the probability of the cardiovascular condition in the subject. The one or more cardiovascular risk scores 110 comprises a first score 110A and a second score 110B. The first score 110A is indicative of the probability of developing the cardiovascular condition in the subject 106. The second score 110B is indicative of the probability of developing the PE in the subject 106. In an embodiment, the first score 110A is indicative of the probability of developing the cardiovascular condition such as the GH in the subject 106. Subsequently, the method 200 terminates at step 214.

Therefore, the method 200 provides a structured and systematic approach to cardiovascular monitoring during pregnancy by sequentially acquiring cardiovascular health data, cardiac output data, and arterial stiffness data, and consolidating these into a composite cardiovascular state representation, thereby ensuring that no critical physiological parameter is overlooked in the assessment of the cardiovascular condition of the subject 106. The method 200 further reduces diagnostic latency by enabling continuous and concurrent acquisition of multiple cardiovascular parameters in a non-invasive manner, thereby facilitating early detection of pregnancy-related cardiovascular complications at a clinically actionable stage, where timely intervention can significantly improve maternal and foetal outcomes. Collectively, the method 200 offers a reliable, scalable, and clinically efficient framework for comprehensive cardiovascular risk assessment during pregnancy, integrating physiological data acquisition, composite state generation, and predictive pattern recognition into a seamless, end-to-end diagnostic workflow.

In a non-limiting embodiment, the present invention relates to a device (not shown) for predicting a cardiovascular condition in a subject during pregnancy. The device forms an integral part of the system 100 as described above, and accordingly, all embodiments, features, and configurations pertaining to the system 100 are equally applicable to the device and are incorporated herein by reference.

The device is configured to acquire cardiovascular health data, cardiac output data, and arterial stiffness data from the subject 106 in a continuous and non-invasive manner, and to consolidate the acquired data into a composite cardiovascular state representation. The device further comprises a trained predictive model that analyses the composite cardiovascular state representation to identify maladaptive haemodynamic patterns and generate a predictive output indicative of the probability of the subject 106 developing the cardiovascular condition such as the PE and the GH.

In one embodiment, the device may be implemented as a wearable, portable, or point-of-care diagnostic unit, thereby enabling continuous cardiovascular surveillance of the subject 106 outside of a conventional clinical environment. In another embodiment, the device may be integrated with a remote monitoring platform, facilitating real-time transmission of physiological data and predictive outputs to a healthcare provider for timely clinical review and intervention. The device thus provides a self-contained, intelligent, and clinically actionable diagnostic solution that combines continuous physiological data acquisition with predictive analytical capability, enabling early and accurate identification of subjects 106 at risk of developing the PE and GH and supporting timely therapeutic intervention to improve maternal and foetal outcomes.

Advantageously, the claimed invention as disclosed above is not routine, conventional, or well understood in the art, as the claimed aspects enable the following solutions to the existing problems in conventional technologies. Specifically, the present invention provides a non-invasive, cost-effective, and accessible solution for predicting cardiovascular conditions, namely Preeclampsia (PE) and Gestational Hypertension (GH), in a subject during pregnancy. Unlike conventional diagnostic methods, such as blood tests for prenatal cell-free DNA, angiogenic marker assays, and urine-based proteinuria detection, the present invention does not require the collection of biological samples, such as blood, urine, or tissue, from the subject, thereby eliminating the associated risks of infection, patient discomfort, and the need for skilled clinical personnel to perform invasive procedures. Further, by utilizing one or more sensors attached to the subject to procure physiological waveform data, and a control unit to derive cardiovascular parameters and generate cardiovascular risk scores, the present system enables continuous, real-time monitoring of the hemodynamic and cardiovascular state of the subject throughout the gestational period, which is otherwise not achievable through conventional laboratory-based diagnostic methods. Additionally, the trained predictive model receives input from the one or more sensors, thereby making the system a multi-modal system.

Furthermore, the use of a photoplethysmography (PPG) sensor, a doppler ultrasonic sensor, and a pump and pressure sensor in the present invention provides a comprehensive and non-invasive means of procuring physiological waveform data of the subject during pregnancy.

Moreover, procuring physiological waveform data comprising blood volume, blood flow, and pulse pressure waveform provides a holistic and clinically meaningful representation of the cardiovascular and hemodynamic state of the subject during pregnancy, without necessitating any invasive procedure or biological sample collection. Additionally, the derivation of a plurality of cardiovascular parameters from the physiological waveform data of the subject provides a significantly more comprehensive and quantitatively precise characterization of the cardiovascular and hemodynamic state of the subject than that afforded by conventional diagnostic methods.

Also, the incorporation of historical medical data of the subject as an input to the trained predictive model significantly enhances the predictive accuracy and clinical relevance of the cardiovascular risk scores generated by the present invention. Further, the combination of cardiovascular health data, cardiac output data, and arterial stiffness data of the subject to generate a composite cardiovascular state representation provides a uniquely comprehensive and clinically meaningful basis for predicting the PE and the GH. Furthermore, the training of the predictive model based on a pre-determined set of cardiovascular conditions corresponding to the probability of developing PE and GH in the subject enables the present invention to perform a clinically significant differentiation between these two closely related but pathophysiologically distinct conditions.

Also, the generation of a first score indicative of the probability of developing a cardiovascular condition, such as GH, and a second score indicative of the probability of developing PE, provides the healthcare provider with a granular, condition-specific, and clinically actionable risk assessment of the subject during pregnancy.

Additionally, the use of a multi-model predictive architecture comprising a Bayesian model, a Tree-Based model, a Deep Learning model, and a Predictive and Discriminative model, operating simultaneously and generating a composite cardiovascular risk score through weighted aggregation of their respective probability outputs, provides a significantly more robust, accurate, and clinically reliable prediction of PE and GH than that achievable through any single predictive model.

In light of the abovementioned advantages and the technical advancements provided by the disclosed system and method, the claimed steps as discussed above are not routine, conventional, or well understood in the art, as the claimed steps provide solutions to the existing problems in conventional technologies. Further, the claimed steps clearly bring an improvement in the functioning of the system itself as the claimed steps provide a technical solution to a technical problem.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable storage medium” should be understood to include tangible items and exclude carrier waves and transient signals, i.e., be non-transitory. Examples include random access memory (RAM), read-only memory (ROM), volatile memory, non-volatile memory, hard drives, CD ROMs, DVDs, flash drives, disks, and any other known physical storage media”.

While the present invention has been described with respect to certain embodiments, it will be apparent to those skilled in the art that various changes and modification may be made without departing from the scope of the invention as defined in the following claims.

### LIST OF REFERENCE NUMERALS

100—System for predicting a cardiovascular condition
102—One or More Sensors
102A—Photoplethysmography (PPG) Sensor
102B—Doppler Ultrasonic Sensor
102C—Pump and Pressure Sensor
102D—Thermistor Sensor
104—Control Unit
106—Subject
108—Display Unit
110—One or More Cardiovascular Risk Scores
110A—First Score
110B—Second Score
200—Method for predicting the cardiovascular condition

1. A system (100) for predicting a cardiovascular condition in a subject (106) during pregnancy, the system (100) comprising:
a control unit (104), the control unit (104) being configured to:
receive one or more physiological waveform data of the subject (106) and a historical medical data of the subject (106);
derive a plurality of cardiovascular parameters based on an analysis of the received physiological waveform data of the subject (106);
process the derived cardiovascular parameters based on a trained predictive model, the predictive model being trained based on a pre-determined set of one or more cardiovascular conditions and the received historical medical data of the subject (106); and
generate one or more cardiovascular risk scores (110) based on the processing, thereby predicting the cardiovascular condition in the subject (106) during pregnancy.

2. The system (100) as claimed in claim 1, comprising one or more sensors (102) being communicably coupled to the control unit (104), the one or more sensors (102) being attached to the subject (106), the one or more sensors (102) being configured to procure the one or more physiological waveform data of the subject (106), wherein the one or more sensors (102) comprise at least one of:
a photoplethysmography (PPG) sensor (102A) being configured to determine changes in blood volume in the subject (106) and color of blood of the subject (106);
a doppler ultrasonic sensor (102B) being configured to determine blood flow in the subject (106);
a pump and pressure sensor (102C) being configured to determine a pulse pressure waveform of the subject (106); and
a thermistor sensor (102D) being configured to determine a blood temperature of the subject (106).

3. The system (100) as claimed in claim 1, wherein the physiological waveform data comprises at least one of: the blood volume, the blood flow, and the pulse pressure waveform.

4. The system (100) as claimed in claim 1, wherein the plurality of cardiovascular parameters comprises at least one of: an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

5. The system (100) as claimed in claim 1, wherein the historical medical data of the subject (106) comprises at least one of: an age of the subject (106), a blood pressure at different gestational ages of the subject (106), a pre-disposition to hypertension of the subject (106), a family history of hypertension of the subject (106), a previous history of preeclampsia of the subject (106), a previous history of gestational hypertension of the subject (106), a presence of diabetes in the subject (106), a history of gestational diabetes in the subject (106), a medication regimen of the subject (106) and a family history of the subject (106).

6. The system (100) as claimed in claim 1, wherein the control unit (104) being configured to:
combine a plurality of a cardiovascular health data of the subject (106), a cardiac output data of the subject (106) and an arterial stiffness data of the subject (106);
generate a composite cardiovascular state representation of the subject (106), based on the combination;
identify a hemodynamic pattern indicative of a maladaptive cardiovascular adaptation during pregnancy, based on the generated composite cardiovascular state representation; and
generate the one or more cardiovascular risk scores (110) based on the identified hemodynamic pattern.

7. The system (100) as claimed in claim 6, wherein the pre-determined set of one or more cardiovascular conditions correspond to at least one of: a probability of developing a cardiovascular condition in the subject (106), a probability of developing Preeclampsia (PE) in the subject (106) and the probability of developing Gestational Hypertension (GH) in the subject (106).

8. The system (100) as claimed in claim 7, wherein the generated one or more cardiovascular risk scores (110) being indicative of the probability of the cardiovascular condition in the subject (106), the one or more cardiovascular risk scores (110) comprise:
a first score (110A), the first score (110A) being indicative of the probability of developing the cardiovascular condition in the subject (106); and
a second score (110B), the second score (110B) being indicative of the probability of developing the PE in the subject (106).

9. The system (100) as claimed in claim 6, wherein the trained predictive model comprises a multi-model predictive architecture configured to:
process the composite cardiovascular state representation using one or more trained predictive models operating simultaneously, the one or more trained predictive models comprising: a Bayesian model, a Tree-Based model, a Deep Learning Model and a Predictive and Discriminative Model;
map the composite cardiovascular state representation, by each of the one or more trained predictive models to a respective probability of the subject (106) developing the cardiovascular condition; and
merge the respective probability outputs of the one or more trained predictive models to generate a composite cardiovascular risk score for the subject (106).

10. The system (100) as claimed in claim 1, comprising a display unit (108), the display unit (108) being communicably coupled with the control unit (104), and the display unit (108) being configured to display at least one of: the physiological waveform data, the plurality of cardiovascular parameters, the historical medical data of the subject (106), and the one or more cardiovascular risk scores.

11. A method (200) for predicting a cardiovascular condition in a subject (106) during pregnancy, the method (200) comprising:
receiving (204), by a control unit (104), one or more physiological waveform data of the subject (106);
receiving (206), by the control unit (104), a historical medical data of the subject (106);
deriving (208), by the control unit (104), a plurality of cardiovascular parameters based on an analysis of the received physiological waveform data of the subject (106);
processing (210), by the control unit, the derived cardiovascular parameters based on a trained predictive model, the predictive model being trained based on a pre-determined set of one or more cardiovascular conditions and the received historical medical data of the subject (106); and
generating (212), by the control unit (104), one or more cardiovascular risk scores (110) based on the processing, thereby predicting the cardiovascular condition in the subject (106) during pregnancy.

12. The method (200) as claimed in claim 11, wherein the physiological waveform data comprises at least one of: the blood volume, the blood flow, and the pulse pressure waveform.

13. The method (200) as claimed in claim 11, wherein the plurality of cardiovascular parameters comprises at least one of: an aortic diastolic blood pressure, an aortic pulse pressure, an aortic systolic blood pressure, an augmentation index, an augmentation pressure, a cardiac index, a cardiac output, an end systolic pressure, an end systolic pressure index, a mean arterial pressure, a pulse pressure stroke volume, a subendocardial viability ratio and a total peripheral resistance.

14. The method (200) as claimed in claim 11, wherein the historical medical data of the subject (106) comprises at least one of: an age of the subject (106), a blood pressure at different gestational ages of the subject (106), a pre-disposition to hypertension of the subject (106), a family history of hypertension of the subject (106), a previous history of preeclampsia of the subject (106), a previous history of gestational hypertension of the subject (106), a presence of diabetes in the subject (106), a history of gestational diabetes in the subject (106), a medication regimen of the subject (106) and a family history of the subject (106).

15. The method (200) as claimed in claim 11, wherein the method (200) comprises the steps of:
combining, by the control unit (104), a plurality of a cardiovascular health data of the subject (106), a cardiac output data of the subject (106) and an arterial stiffness data of the subject (106);
generating, by the control unit (104), a composite cardiovascular state representation of the subject (106), based on the combination;
identifying, by the control unit (104), a hemodynamic pattern indicative of a maladaptive cardiovascular adaptation during pregnancy, based on the generated composite cardiovascular state representation; and
generating, by the control unit (104), the one or more cardiovascular risk scores (110) based on the identified hemodynamic pattern.

16. The method (200) as claimed in claim 15, wherein the pre-determined set of one or more cardiovascular conditions correspond to at least one of: a probability of developing a cardiovascular condition in the subject (106), a probability of developing Preeclampsia (PE) in the subject (106) and the probability of developing Gestational Hypertension (GH) in the subject (106).

17. The method (200) as claimed in claim 16, wherein the generated one or more cardiovascular risk scores (110) being indicative of the probability of the cardiovascular condition in the subject (106), the one or more cardiovascular risk scores (110) comprise:
a first score (110A), the first score (110A) being indicative of the probability of developing the cardiovascular condition in the subject (106); and
a second score (110B), the second score (110B) being indicative of the
probability of developing the PE in the subject (106).

18. The method (200) as claimed in claim 15, wherein the method (200) comprises the steps of:
processing, by the control unit (106), the composite cardiovascular state representation using one or more trained predictive models operating simultaneously, the one or more trained predictive models comprising: a Bayesian model, a Tree-Based model, a Deep Learning Model and a Predictive and Discriminative Model;
mapping, by the control unit (106), the composite cardiovascular state representation, by each of the one or more trained predictive models to a respective probability of the subject (106) developing the cardiovascular condition; and
merging, by the control unit (106), the respective probability outputs of the one or more trained predictive models to generate a composite cardiovascular risk score for the subject (106).
